(19) United States
(12) Patent Application Publication
(10) Pub. No.: US 2018/0325722 A1
(43) Pub. Date: Nov. 15, 2018

(54) MOUTHGUARD

(71) Applicants: Jeff Weiss, Canoga Park, CA (US); John Scalco, Canoga Park, CA (US)

(72) Inventors: Jeff Weiss, Canoga Park, CA (US); John Scalco, Canoga Park, CA (US)

(21) Appl. No.: 15/972,631

(22) Filed: May 7, 2018

Related U.S. Application Data
(60) Provisional application No. 62/506,503, filed on May 15, 2017.

(51) Int. Cl. A61F 5/56; A63B 71/08

(52) U.S. Cl. CPC A61F 5/566; A61F 2005/563; A63B 71/085

(57) ABSTRACT

The present invention discloses a mouthguard that conforms to the dental configuration of a wearer to protect from bruxism or grinding of teeth. The mouthguard comprises at least one U-shaped channel with an inner member attached to an outer member, and at least one protrusion is provided on integrally extends from a central front and a central rear portion of the at least one channel. The inner member is made of deformable material and the outer member is made of flexible material. The U-shaped channel is configured to receive an upper or lower row of teeth of the wearer. The protrusion is configured to act as a cushion contact when the upper and lower teeth are moved towards one another. The mouthguard could be remolded by the wearer, based on their requirement or comfort.

### BACKGROUND OF THE INVENTION

### A. Technical Field

The present invention generally relates to a mouthguard for protecting teeth from bruxism. More specifically, the present invention relates to the mouthguard that conforms to the dental configuration of a wearer or a user.

### B. Description of Related Art

Generally, the behavior of clenching and grinding of teeth is called bruxism. To be precise, it is an unconscious behaviour or habit, where people tightly hold their top and bottom teeth together or slide the teeth back and forth over each other, performed to release anxiety, aggression or anger. Teeth grinding can create numerous problems such as local muscular pain, headaches, loss of tooth structure, gum recession, loose teeth, shortening of teeth, tooth sensitivity, cracked and broken teeth, damage to the bone structure of the jaw joint and even results in facial changes. To address the foregoing issues, mouthguards have been developed. Mouthguards are affixed either to the upper or lower jaw teeth to protect the teeth, reduce the chance of shock, concussions and other injuries resulting from high impact collisions and blows due to the unconscious teeth grinding.

Currently, various types of mouthguards are manufactured and available in the market, which includes non-personalized mouthguards, universal and stock model type mouthguards, or a custom formed mouthguard to the teeth of an individual wearer. However, personalized and/or proper fit mouthguards require the services of a dentist or other type of skilled practitioner, which adds significant cost to the mouthguard. In addition to the cost, they are bulky and result in breathing and speaking difficultly.

Mouthguards are generally made of a single rigid material, which can cause discomfort for the gums, teeth, and jaw of the user. It doesn't provide sufficient cushioning, protection form malocclusion conditions, when the user bites or grinds the teeth. Hence, these mouthguards are only partially successful and not often used. Further, some mouthguards comprise a smaller configuration, which may not provide a better fit inside the mouth, where there is a danger of being swallowed accidentally. As will be appreciated, with all of the above mouthguards, there are many complaints from patients regarding the mouthguard fitting well in their mouths, and children will rarely ever wear them due to the ill-fitting nature of the mouthguard as well as other inconveniences in their use.

Therefore, there is a need to provide an efficient mouthguard design that could be constructed using different deformable materials to prevent bruxism effectively. There is also a need to provide a mouthguard with a better fit to prevent unexpected falls from the user's mouth. There is also a need to provide a mouthguard which provided more comfort to the wearer. There is also a need to provide a mouthguard with protrusions to provide protection from overbite and underbite. Further, there is also a need for a mouthguard that provides sufficient cushioning during unconscious clenching and grinding of teeth.

### SUMMARY OF THE INVENTION

The present invention discloses a mouthguard or mouth protecting device to protect teeth from bruxism and forming a mouthguard that conforms to the dentition of a wearer or a user.

According to the present invention, the mouthguard comprising at least one channel with an inner member attached to an outer member and protrusions, which integrally extends from a central front and a central rear portion of the channel. The channel is of U-shaped design, which is configured to receive an upper or lower row of teeth of the wearer. In on embodiment, the protrusion acts as a cushion contact when the upper and lower teeth are moved toward one another, to provide protection from malocclusion conditions. The mouthguard is adapted to be moldable to conform over the dental arc of the wearer. One channel is adapted to extend at least partially over the dental arch. The mouthguard could be remolded by the wearer if a better fit is required, or because of teeth shift.

In one embodiment, the inner member of the mouthguard is made of soft deformable materials. In another embodiment, the inner member is formed from one or more materials from a group comprising co-polymer, ethylene vinyl acetate (EVA) copolymers, polystyrene, polyurethane and mix of copolymer materials. The outer member is made of flexible materials. In another embodiment, the outer member is formed from one or more materials from a group comprising thermoplastic elastomers (TPE), thermoplastic rubbers, and mix of polymers. In one embodiment, the thickness of the inner layer is greater than that of a thickness of an outer layer. In one embodiment, the mouthguard provides a snug fit over the dental arc of the wearer by keeping it in hot water.

In one embodiment, a method of molding a mouthguard by wearer to a dentition of a wearer is disclosed. At one step, a mouthguard comprising, a U-shaped channel with an inner member attached to an outer member is provided. The mouthguard is configured to conform onto the upper or lower teeth of the wearer. At second step, at least one protrusion at a central front and other protrusion at a central rear portion of the U-shaped channel is provided. At third step, conforming the U-shaped channel by applying hot water to a shape, which conforms to a dental arch of the wearer.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The embodiments herein will be better understood from the following detailed description with reference to the drawings, in which:

FIG. 1 is a front perspective view of the mouthguard in an embodiment of the present invention.

FIG. 2 is a rear perspective view of the mouthguard in an embodiment of the present invention.

FIG. 3 is a top view of the mouthguard in an embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

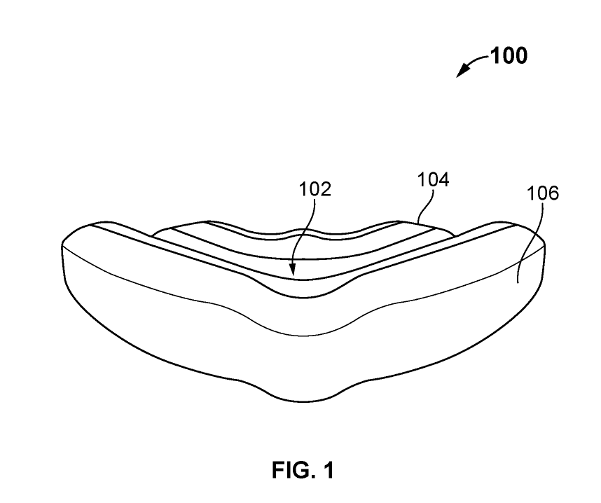

Referring to FIG. 1, a front view of a mouthguard 100 according to an embodiment of the invention. The mouthguard 100 comprises at least one channel/U-shaped channel 102 with an inner member 104 attached to an outer member 106, and at least one channel 102 is configured to receive an upper or a lower row of teeth of a wearer.

In one embodiment, the inner member 104 is made of soft deformable layer. In another embodiment, the inner member is formed from one or more material from a group comprising co-polymer, ethylene vinyl acetate (EVA) copolymers, polystyrene, polyurethane and mix of copolymer materials. In one embodiment, the outer member 106 is made of flexible materials. In another embodiment, the outer member is formed from one or more material from a group comprising thermoplastic elastomers (TPE), thermoplastic rubbers, and mix of polymers thereof.

In another embodiment, the inner member 104 is formed by a layer of thermoplastic material such as ethyl vinyl acetate (EVA) that encloses and encapsulates the outer section walls 104. EVA softens at temperature in range from 85° C. to 95° C. This property enables the mouthguard to be shaped to conform to the arch and teeth of the wearer.

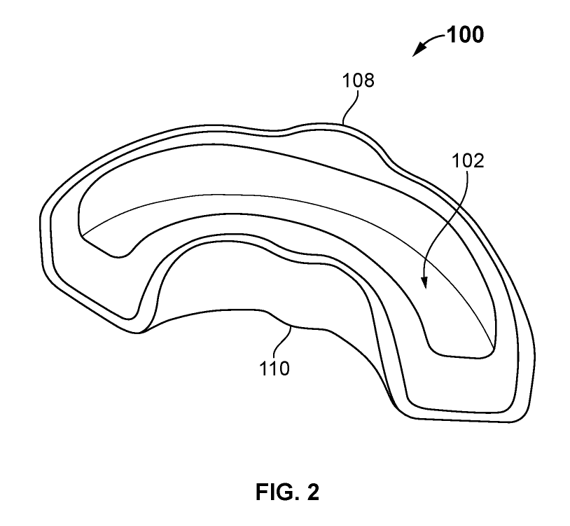

Referring to FIG. 2, a rear perspective view of the mouthguard 100, is illustrated. The mouthguard 100 comprises at least one protrusion 108 that integrally extends from a central front, and other protrusion 110 extends from a central rear portion of the at least one channel 102. The protrusions 108, 110 are configured to act as a cushion contact when the upper and lower teeth are moved toward one another. The mouthguard protrusions 108, 110 also provide protection from malocclusion conditions such as overbite, underbite and misalignment or incorrect relation between the teeth of the two dental arches.

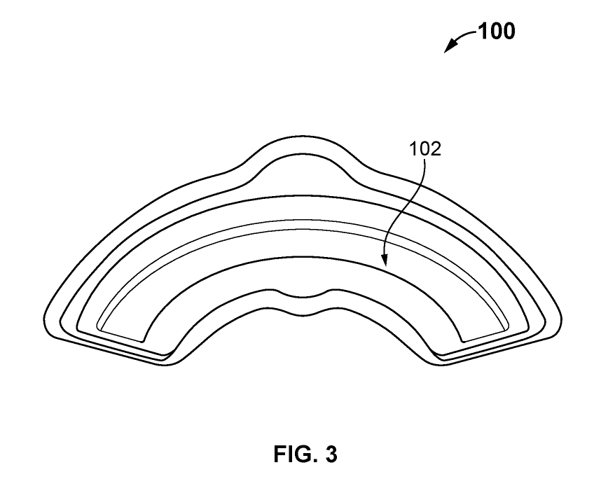

Referring to FIG. 3, a top view of the mouthguard 100, which has a broadly U-shaped channel 102 within the inner member side walls, is illustrated. The U-shaped channel 102 may be a mandibular arch or a dental arch. In another embodiment, the mandibular arch 102 adapts the wearer's teeth to fit correctly into it. The mandibular arch 102 is formed with a soft material so that the wearer or user could feel more comfort while sleeping.

Another benefit of the present invention, the mouthguard of the teeth will shape and mold accordingly, by application of the pressure exerted by the jaws upon biting down by the wearer. In addition to the performance benefits, athletes and patients can easily use the mouthguard to a shape that conforms to the contours of the mouth.

The mouthguard 100 not only tends to eliminate the problem of the bruxism effect or teeth grinding but also helps in keeping the mouth open. This configuration also creates an air passage to make breathing easier for the user, in some instances, during sleeping.

In one embodiment, a method of molding a mouthguard 100 by wearer to a dentition of a wearer is disclosed. At one step, a mouthguard comprising, a U-shaped channel 102 with an inner member 104 attached to an outer member 106 is provided. The mouthguard 100 is configured to conform onto an upper or a lower tooth of a wearer. At the second step, at least one protrusion 108 at a central front and other protrusion 110 at a central rear portion of the U-shaped channel 102 is provided. At the third step, conforming the U-shaped channel 102 by applying hot water to a shape, which conforms to a dental arch of the wearer.

Advantageously, the present mouthguard 100 is more comfortable and provides flexibility to the wearer. The mouthguard 100 facilitates a better fit inside the wearer's mouth by means of snug fit. The snug fit prevents unexpected falls of the mouthguard 100 from wearer's mouth. The protrusions 108, 110 of the mouthguard 100 provides cushion and protection from the malocclusion conditions such as overbite, underbite and misalignments between wearer's teeth. The mouthguard 100 could be remolded by the wearer, in some instances, if a better fit is required or because of teeth shift. The invention provides sufficient cushion when the wearer bites down or grinds the teeth during sleep time.

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in a generic and descriptive sense and not for purposes of limitation. Accordingly, the present invention is not limited to the specific embodiments illustrated herein.

What is claimed is:

1. A mouthguard, comprising:
at least one channel with an inner member attached to an outer member, the channel is configured to receive an upper or a lower row of teeth of a wearer; and
at least one protrusion integrally extends from a central front and a central rear portion of the channel is configured to provide a cushion contact to the wearer's teeth,
wherein said mouthguard is adapted to be moldable to conform over the dental arc of the wearer.

2. The mouthguard of claim 1, wherein the channel comprises a U-shaped channel.

3. The mouthguard of claim 1, wherein the channel is adapted extend at least partially over the dental arch.

4. The mouthguard of claim 1, wherein the inner member is made of soft deformable material.

5. The mouthguard of claim 1, wherein the outer member is made of flexible material.

6. The mouthguard of claim 1, wherein the outer member is made of one or more material from a group comprising thermoplastic elastomers (TPE), thermoplastic rubbers, and mix of polymers.

7. The mouthguard of claim 1, wherein the inner member is made of one or more materials from a group comprising co-polymer, ethylene vinyl acetate (EVA) copolymers, polystyrene, polyurethane and mix of copolymer materials.

8. The mouthguard of claim 1, wherein the inner layer comprises thickness greater than that of a thickness of an outer layer.

9. The mouthguard of claim 1, wherein the mouthguard is adapted to be wearer conformable in hot water to provide snug fit over the dental arc of the wearer.

10. A method of molding a mouthguard, comprising the steps of:
providing a mouthguard comprising,
a U-shaped channel with an inner member attached to an outer member to conform onto an upper or lower tooth of a wearer, and
at least one protrusion at a central front and a central rear portion of the U-shaped channel; and
conforming the U-shaped channel in shape to a dental arch of the wearer by applying hot water.
